(19) United States
(12) Patent Application Publication
(10) Pub. No.: US 2009/0149924 A1
(43) Pub. Date: Jun. 11, 2009

(54) TREATMENT OF EAR INFECTION USING HANDS-FREE BLUE/VIOLET LIGHT DEVICE

(76) Inventor: Susan Lemons White, Raleigh, NC (US)

Correspondence Address: PASSE' INTELLECTUAL PROPERTY, LLC, 1717 BRASSFIELD RD., RALEIGH, NC, 27614, US

(21) Appl. No.: 11/952,061

(22) Filed: Dec. 6, 2007

(51) Int. Cl. A61B 18/18

(52) U.S. Cl. 607/94

(57) ABSTRACT

The present invention relates to the treatment of Otis infection of the ear or other infection of the ear by application of blue violet light. The device of the present invention operates hands free mounting over the ear in such a way to provide light treatments without the need to hold the device by positioning the light source such that the light is positioned to shine into the ear canal.

### COPYRIGHT NOTICE

A portion of the disclosure of this patent contains material that is subject to copyright protection. The copyright owner has no objection to the reproduction by anyone of the patent document or the patent disclosure as it appears in the Patent and Trademark Office patent files or records, but otherwise reserves all copyright rights whatsoever.

### BACKGROUND OF THE INVENTION

1. Field of the Invention

The present invention relates to the treatment of ear infections. More specifically the present invention relates to the treatment of ear infections with a blue/violet light hands-free device.

1. Description of Related Art

Otitis media is an inflammation of the middle ear, the space behind the ear drum. It is one of the two conditions that are commonly thought of as ear infections, the other being Otitis externa. Ear infections are very common in childhood, and includes acute and chronic conditions; all of which involve inflammation of the ear drum (tympanic membrane), and are usually associated with a buildup of fluid in the space behind the ear drum (middle ear space).

Inflammation of the skin of the ear canal is the essence of all Otitis infections. If inflammation progresses to infection, the ear canal may fill with swollen tissue and drainage. Once the ear canal is blocked, hearing will be dampened (conductive hearing impairment) until the condition improves. In very severe cases, the skin infection can spread to the face (facial cellulitis) and to the major salivary gland in the cheek (parotitis). In that situation, moving the jaw and eating become painful. In its mildest forms, external Otitis is so common that some ear, nose and throat physicians have suggested that most people will have an episode at some point in life. In many individuals, for the reasons discussed below, the condition is recurrent and will happen several times in a lifetime (chronic Otitis).

The use of topical solutions and suspensions in the form of ear drops and ear spray are the current mainstay of treatment for Otitis. These drops both physically wash collected debris, shed skin and infected drainage from the ear canal, and contain substances that either kill pathogenic germs, stop them from multiplying, or do both. The drops generally contain drying substances (astringents), acidifying agents, antibiotics and/or anti-fungal agents. Some prescription drops also contain anti-inflammatory steroids. Although there is evidence that steroids are effective at reducing the length of treatment time required, fungal Otitis may be aggravated by the use of topical steroids.

When the condition has progressed to the point where the ear canal is blocked, a physician may have to begin treatment by clearing the ear under otoscopic examination and placing a thin strip of an absorbent material (ear wick) into the ear canal. In severe cases of Otitis, an otologist is needed to carefully clean out the ear canal under microscopic visualization. In such severe cases, in which drainage is abundant enough to recurrently block the ear canal, a qualified health professional may aspirate the ear as many times as twice a week for the first two or three weeks of treatment. It is imperative that there is visualization of an intact tympanic membrane. Use of certain medications with a ruptured tympanic membrane can cause tinnitus, vertigo, dizziness and hearing loss in some cases.

Although the acute infection of external Otitis generally resolves in a few days with topical washes and antibiotics, it normally takes weeks before the ear canal skin is fully normal. The glands of the outer skin of the ear canal will not begin producing cerumen again until the skin is not only no longer infected, but no longer inflamed. Once healed completely, the ear canal is again self-cleaning. Until then, slight irritation can be enough to cause external Otitis to flare again.

As stated above, effective medications include ear drops or sprays containing antibiotics to fight infection, and corticosteroids to reduce itching and inflammation. The first line is currently a topical preparation such as 2% acetic acid or a topical antibiotic solution containing antibiotics such as aminoglycoside, polymyxin or fluoroquinolone. It is possible to have both a bacterial and fungal ear infection, and many of the topical treatments are designed to cure both.

Occasionally, pills may be used in addition to the topical medications. Analgesics may be used if pain is severe. Putting something warm against the ears may reduce pain.

Light of various frequencies has been used to treat various conditions including acne and various bacterial infections on the face and throat. In US published patent application 2003/0009158 discloses skin treatments using blue and violet light. Aging or damaged skin is treated by irradiating affected skin areas with an effective amount of light. The light can be from a light source or from sunlight. In addition, treatments of the skin with light and compounds or compositions which enhance light penetration are disclosed.

In U.S. Pat. No. 6,953,341 to Black issued Oct. 11, 2005 there is disclosed a “toothpick” for the light treatment of body structures. The device has a handle and a tapered element for delivering light of various frequencies. As the name toothpick implies, the device is designed essentially for use as an oral hygiene device. While a number of uses are listed there are no examples of the device using any particular light frequency or that any particular disease is treatable with the device. In addition, the device comprises a massaging means for massaging the gums or other oral structures. The large size and tapered nature of the device as well as the vibratory nature of the device make it unsuitable for sensitive structures such as the ear and useful only for oral or like structures. Even further, the design would allow a user to accidently insert the device into the ear canal and potentially injure the ear drum. Further, the device provides not only blue and violet light it provides green light as well as a means for using the device to massage the body structure the device is used on.

In U.S. Pat. No. 5,292,346 to Ceravolo issued Mar. 8, 1994 there is described a bactericidal ultraviolet light radiating device for the treatment of mucosal or dermal tissues having a light source, optical light directing lens coupled to the light source and an electric power supply to power the light source. The device is described as intended for oral therapeutic radiation application of ultraviolet light (below about 400 nanometers in wavelength). While it is described as being able to be used in the ear no discussion of the effectiveness of the design or of actual use in the ear is described.

In U.S. Pat. No. 4,865,035 to Mori, there is described a device which delivers the entire visible spectrum of light designed to be close to the entire suns visible spectrum. The invention is described as safer than use of ultraviolet light however requires use of a long probe light source which could be detrimental if inserted in the ear but necessary to deliver natural light of this particular invention. No indication that broad spectrum light does anything to treat ear infections and in fact broad spectrum light natural light does not appear to work as a cure for Otitis.

Use of blue/violet light has been well documented as useful in the treatment of skin conditions such as acne vulgaris. It has been demonstrated that the main bacteria involved in acne the P acnes bacteria which is the cause of the acne skin lesions is sensitive to this light range. For example in US patent publication 2004/0122492 to Harth, et al, there is described a large device for combining both blue/violet and IR light to treat skin conditions and in US patent publication 2004/0176823 to Island et al there is described a novel device for the treatment of acne using a blue/violet diode light source.

Accordingly there is a need in the art for new and useful treatments for ear infections other than the presently known treatments.

### SUMMARY OF THE INVENTION

It has been surprisingly discovered that a treatment of an infection of the ear canal, even in individuals who suffer from chronic or persistent middle ear infections, can effectively be treated with an exposure of a concentrated source of light consisting of blue/violet light into the ear canal which is positioned at the opening of the ear canal, and preferably is battery operated and hands-free.

Accordingly, in one embodiment of the invention there is disclosed a device for the treatment of an ear infection in a human suffering from an infection comprising:
a) a housing comprising a guarded LED having a frequency of between about 400 and 500 nanometers and a self contained DC power source for engaging the LED;
b) a means for positioning and holding the housing hands free on the human such that the LED is positioned at the opening of the ear canal of the infected ear;
c) a means for turning the LED on and off.

In another embodiment of the invention there is disclosed a device for the treatment of an ear infection in a human suffering from an infection comprising:
a) a housing comprising a guarded LED having a frequency of between about 400 and 500 nanometers, a means for centering the LED in an ear canal opening and a self contained DC power source for engaging the LED;
b) a means for holding the housing hands free on the human;
c) a means for turning the LED on and off.

These and other objects of the present invention will be clear when taken in view of the detailed specification and disclosure in conjunction with the appended figures.

### BRIEF DESCRIPTION OF THE DRAWINGS

FIG. 1 is a perspective view of an embodiment of the invention with an ear hanging hook.

FIG. 2 is a perspective view of a device of the present invention on a human using a head strap.

FIG. 3 is a top angle perspective of a device of the invention showing ear canal centering knobs.

FIG. 4 is a cut through perspective of an embodiment of the invention.

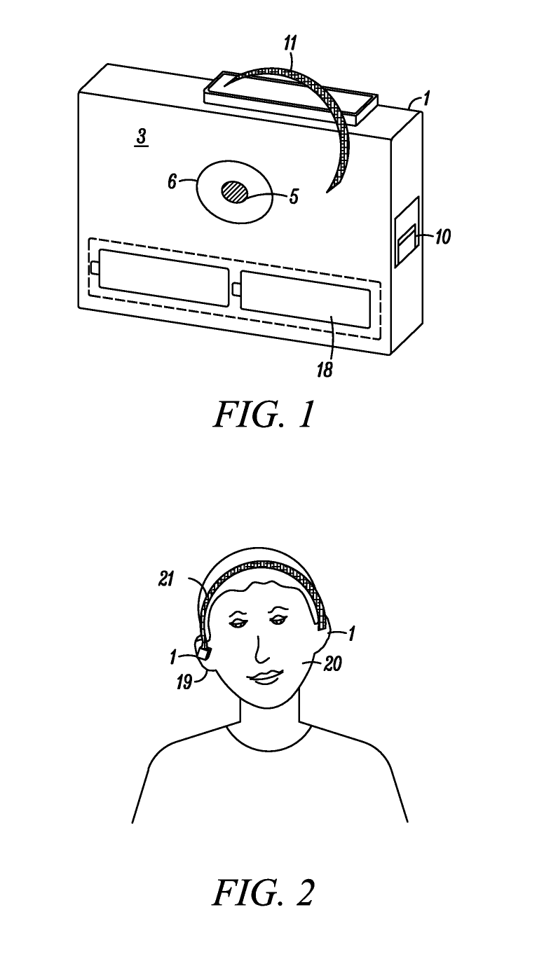

### DETAILED DESCRIPTION OF THE INVENTION

The general description of the invention and how to use the device of the present invention is stated in the Brief Summary above. This detailed description defines the meaning of the terms used herein and specifically describes embodiments in order for those skilled in the art to practice the invention. The above interests in successfully treating ear infections can be seen from the disclosure which follows and are met by the present invention.

As used herein “ear infection” relates to an infection of some portion of the ear canal of a human. This, in general, will be any portion of the ear canal e.g. middle, exterior, etc, that the light of the present invention can penetrate to, that has been infected by an organism either bacterial or fungal. The organism would, in general, be an organism that is typical of the Otitis type infections of the ear. Since the light of the invention is positioned at the opening to the ear canal, light from a device will penetrate the exterior ear canal most effectively followed by the middle and inner ear canal. Adjustments in intensity of the light source for the present invention can be made to most effectively treat the infection based on the position of the infection within the ear canal.

As used herein the phrase “administering an effective amount” means to expose the light of the present invention device to the ear canal for sufficient length time, sufficient number of times and in amounts and energy levels sufficient to provide an observable improvement in the ear infection either by a reduction, elimination or a decrease in the time of the infection. One skilled in the art in view of this disclosure could, without undue experimentation select LED sources of different wattages, voltages, intensities and the like consistent with the disclosure herein. In one embodiment a low wattage (between a fraction of a watt and 12 watts) blue LED is selected. In one embodiment the LED has a frequency of between about 450 and about 475 nanometers.

“Sufficient” amounts of light and energy can be delivered by a single exposure to the present invention of a given time or multiple exposures of fixed or varying length of time depending on the position of the infection in the canal, as well as the intensity of the infection. In one embodiment a single exposure is from 5 to 10 minute. Multiple exposures are from about 2 to 5 times daily. In another embodiment for the treatment of chronic ear infection the treatment is extended up to between about 45 to 60 minutes at least one time daily.

As used herein “light” refers to an artificial LED source of light of the frequency of the present invention namely, consisting of about 400 to about 500 nanometers. This can be one LED or a plurality of LEDs as desired. The frequency of a given light source of the present invention can be across the entire range, a portion of the range or can be limited to a single frequency within the range. Since the light is positioned very close to a sensitive body structure, the ear, the light source should be one that does not generate large amounts of heat when in use nor should the light source bulb be excessively large. In addition the bulb should not if at all extend into the ear canal more than about a quarter to a maximum of about a half an inch i.e. a guarded LED. The source of the light in one embodiment is a “blue LED”. The blue LED is known to be within the range necessary for treatment in the present invention, does not generate large amounts of heat, is relatively small and can deliver light to the ear canal without fear of the damaging effects of a larger bulb or many other light sources. In addition, LED bulbs can be made into hands free battery operated units. In one embodiment the LED light bulbs have a wattage of between about a quarter watt to about 12 watts and a frequency output in the range of about 450 to 475 nanometers.

As used herein “treatment of an ear infection” refers to the action of the light in the range of the present invention upon treatment of the ear canal to significantly increase the death rate or completely kill the organisim(s) responsible for an ear canal infection.

By “hands free” as used herein, refers to a battery operated light source, such as a battery operated LED, which is designed to hang on the ear with the light source positioned at the opening of the ear canal. The light would provide an effect without having to hold the device, move it about or be connected to a power source. This could be accomplished, in one embodiment, with a battery operated LED device designed t be positioned with the LED facing the ear canal opening. In another embodiment the device covers the entire ear canal so that light does not leak out of the ear canal and a maximum amount of energy is directed or reflected off the device into the ear canal. In one embodiment hands free is achieved by use of an ear hook which hangs the device on the ear in such a manner that the LED is positioned at the opening of the ear canal. In another embodiment the hands free means is achieved by the use of a over the ear or over the head type head set or head band which, while large, distributes the weight of the device across a larger surface. In yet another embodiment the device is designed with two light sources and is capable of treating both ear canals at once. One example of the dual treatment would be a dual headset type apparatus.

As used herein the term “housing” refers to the container which holds the LED bulbs as well as the power source either directly or indirectly as explained further in this application. The housing can be made of any durable material such as plastic wood metal or the like and can be of any general shape. In one embodiment the housing is a box of a rectangular or other shape while in another embodiment the housing is shaped in the form of ear head phones as shown in FIG. 4 which follows. One skilled in the art could fashion any number of different shapes but in general the LED's must be positioned in the housing to shine the light into the ear canal. Because the ear is relatively sensitive the LED should be positioned right at the opening or no more than about a quarter to a half inch into the canal in a manner that it cannot, even accidently go further into the ear canal and potentially injure the ear drum. In one embodiment the LED is flush with the surface of the housing and in an optional embodiment a protective clear cover is placed over the bulb to prevent any contact with the bulb. In another embodiment the LED sticks out no more than a half inch from the surface of the housing and into the ear canal.

As used herein a “self contained DC power source” refers to a power source which is not corded such as an AC to DC converter. In other words battery operated solar electric operated or the like would work. The power source would include the necessary electronics wires and the like including an on off switch to light the LED merely by turning the switch on. In one embodiment the housing also includes a timer for turning the LED on or off.

As used herein a “means for positioning and holding the housing hands free” refers to the ear or head set/strap described above or any other means using hook and loop, etc to hold the device over the ear. As used herein positioning at the opening of the ear canal refers to positioning the device such that the light from the LED shines into the ear canal. This can be done in simple versions by using the hands free means to manually position the light accordingly. In another embodiment a means to keep the device centered could be used. Versions of this embodiment include nubs that are positioned within the crevices of the ear to prevent forward or backwards movements or use of an over the ear, ear phone design or the like.

The means for turning the LED on or off can be a simple switch or the like. In the case of a solar panel taking the panel out of the box might activate the light. One skilled in the art can determine how to turn the device on or off in view of the disclosure herein.

In use the present invention method would be accomplished as follows. After determining that the ear of a particular human patient is infected, such as by visual observation, description of symptoms or by known culturing methods the present invention would be commenced. A device with a blue/violet light source (and no other light frequencies), would be selected and positioned at the opening of the ear canal. The light source would be positioned such that it faced directly into the ear canal. In one embodiment there is a focusing lens to focus the light toward the ear canal. The device would then be turned on and the ear canal bathed in blue/violet light for the period of time selected based on the location and intensity of infection.

The light would remain on for a selected period of time and the treatment could be repeated either for a fixed number of treatments or repeated treatments could be accomplished and repeated until the infection is partially or completely cured. The cured would be evidenced by a reduction or elimination of symptoms including inflammation. In one embodiment the present treatment of the invention is further combined with known pharmaceutical treatments such as drops, antibiotics or the like to act faster and more thoroughly than a pharmaceutical treatment which is currently the only method of treating ear infections. In some embodiments the light treatment alone is sufficient.

Now referring to the drawings, Figure one is a perspective view of a device 1 of the present invention. This device has ear hook 11 for hanging the device 1 onto the affected ear of a patient. Shown are a housing 3 which contains a Blue LED 5 covered by lens 6. In this view we can see inside housing 3 to see DC power source batteries 18 which power the LED 5. The LED 6 is turned on by switch 10 which delivers power from batteries 18 to light the LED 6. This unit is positioned so that when hung on the patient's ear the LED is positioned over the ear canal and the LED 6 can shine into the canal. Since the LED 5 is behind lens 6 it rests just at the outside of the ear canal and not in the ear canal in any way.

In FIG. 2 a perspective of the present invention is shown with a device 1 shown against the ear 19 of patient 20. The device 1 is held in place in this embodiment via head strap 21. In this embodiment the LED 5 not shown is facing the ear canal of the patient 20, the switch turned on the light from the LED 5 shining into the ear canal of patient 20. This version could simply be modified to consist of two LED's one for each ear.

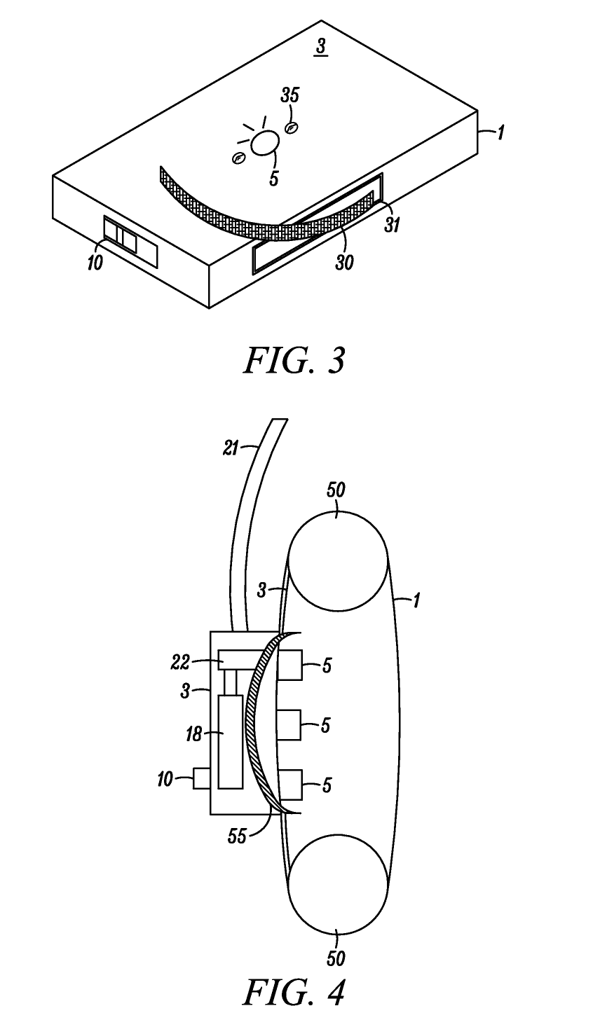

FIG. 3 is another embodiment of the present invention device 1. In this perspective an ear hook 30 is shown for hanging the device over the ear of the patient and also a hinge 31 enabling the ear hook 30 to be swiveled and therefore used on either ear. The LED 5 in this perspective is not behind a lens rather it sticks up from the surface of the housing 3 about a quarter inch. Thus when this embodiment of device 1 is place over the ear the LED 5 is positioned slightly in the ear canal. In this view are also shown ear nubs 35. The ear nubs 35 are designed to sit inside the other portions of the external ear and prevent device 1 from moving forward or backwards thus further aiding the maintaining of the LED at the opening of an ear canal in use.

In FIG. 4 we have an embodiment of the device 1 which is a cut through perspective of an over the ear, ear phone type design. In this embodiment there is a plurality of LEDs 5 positioned in housing 3. Another feature is shown in this perspective, that of reflector 55 which helps focus the light from the 3 LED's 5 toward an ear canal the device is shown with ear foam 50 which forms the part of the housing 3 that fits against the patient around the ear. Shown in the housing again are batteries 18 and in this view a circuit board 22 which can contain timers, LED circuits, or other optional digital features. The over the ear device 1 is held in place by head strap 21 similar to FIG. 2 and only shown as a portion of the head strap 21. In this version the LEDs 5 are held back a bit from the opening but the light tight over the ear feature as well as the reflector help focus a large amount of light down the ear canal in use.

As can be seen the present device is designed to be simple to use, hands free as well as designed to not endanger the inner ear from coming into contact with the present invention device. This represents a device which has the capability of delivering the greatest amount of light to the source of an ear infection, cure the infection and not risk any damage to the ear or ear drum due to sticking a device into the ear more than a safe distance.

### EXAMPLE

A 53 year old female suffering from chronic ear infections was determined to have an infection of the middle ear. A device of the present invention having a blue LED (0.71 lumens) was positioned at the opening of the ear canal and the light turned on. Light treatment consisted of treatment to the ear canal for a period of 20 minutes. Treatment of the canal was repeated at least 3 times per day for a period of 48 hours.

While the particular patient had suffered for years with persistent chronic outer and middle ear infections where each infection lasted several days, after treatment with the method of the invention using a blue LED the infection was completely eradicated within 48 hours after the first treatment.

The above description and example are for the purpose of teaching the person of ordinary skill in the art how to practice the present invention and it is not intended to detail all those obvious modifications and variations of it which become apparent to the skilled worker upon reading the present invention description. It is intended, however, that all such modifications and variations be included within the scope of the present invention, which is defined by the following claims. The claims are intended to cover the claimed steps and components, in any sequence, consistent with the present invention which is effective to meet the objectives herein intended unless the context specifically indicates to the contrary.

What is claimed is:

1. A device for the treatment of an ear infection in a human suffering from an infection comprising:
a) a housing comprising a LED having a frequency of between about 400 and 500 nanometers and a self contained DC power source for engaging the LED;
b) a means for positioning and holding the housing hands free on the human such that the LED is positioned at the opening of the ear canal of the infected ear;
c) a means for turning the LED on and off.

2. A device according to claim 1 wherein the means for positioning and holding the device is a head strap.

3. A device according to claim 1 wherein the means for positioning and holding the device is an ear hook.

4. A device according to claim 1 where the means for positioning and holding the device is an over the ear, ear phone type device.

5. A device according to claim 1 which further comprises a timer for turning the device off.

6. A device according to claim 1 wherein the self contained DC power source is one or more batteries.

7. A device according to claim 1 wherein the self contained DC power source is a solar panel.

8. A device according to claim 1 wherein the means for turning the LED on and off is a switch.

9. A device according to claim 1 wherein the LED further comprises a curved reflector for focusing the light from the LED into the ear canal.

10. A device according to claim 1 wherein there are a plurality of LEDs.

11. A device according to claim 1 wherein the LED is a blue LED.

12. A device according to claim 1 which further comprises a means to guard the bulb from contact with the ear.

13. A device according to claim 12 wherein the guard is a lens over the bulb.

14. A device for the treatment of an ear infection in a human suffering from an infection comprising:
a) a housing comprising a LED having a frequency of between about 400 and 500 nanometers, a means for centering the LED in an ear canal opening and a self contained DC power source for engaging the LED;
b) a means for holding the housing hands free on the human;
c) a means for turning the LED on and off.

15. A device according to claim 14 which further comprises a means to guard the bulb from contact with the ear.

16. A device according to claim 15 wherein the guard is a lens over the bulb.
